(19) United States
(12) Patent Application Publication
(10) Pub. No.: US 2026/0069112 A1
(43) Pub. Date: Mar. 12, 2026

(54) MANUFACTURABILITY IMPROVEMENTS FOR LAPAROSCOPIC INTRODUCER

(71) Applicants: Baxter International Inc., Deerfield, IL (US); Baxter Healthcare SA, Glattpark (Opfikon) (CH)

(72) Inventor: Spencer Scott Paris, Chicago, IL (US)

(21) Appl. No.: 19/323,575

(22) Filed: Sep. 9, 2025

Related U.S. Application Data
(60) Provisional application No. 63/692,443, filed on Sep. 9, 2024.

(51) Int. Cl. A61B 1/00; A61B 1/313; A61B 17/34

(52) U.S. Cl. CPC A61B 1/00142; A61B 1/00154; A61B 1/3132; A61B 17/3423

(57) ABSTRACT

An introducer assembly for applying a film at a surgical site includes a winding rod with a shaft that extends from a handle. The handle is located at a proximal end of the winding rod and includes a plurality of raised protrusions. The shaft includes a slit located at its distal end. The shaft of the winding rod has a dumbbell-shaped cross-section. The introducer assembly also includes an outer cannula assembly with a metallic tube coupled to a molded grip located at the proximal end of the metallic tube. The metallic tube includes a slit at its distal end and a notch at its proximal end; the notch is configured to mate with a tab on the molded grip for rotational clocking. The molded grip includes at least one substantially flat surface to prevent rolling, and includes a plurality of ribs radiating from the surface of the molded grip.

This application claims priority to and the benefit as a non-provisional application of U.S. Provisional Patent Application No. 63/692,443, filed September 9, 2024, the entire contents of which are hereby incorporated by reference and relied upon.

### BACKGROUND

After a surgical procedure, organs and/or tissues adjacent to a surgical site may adhere to the surgical site by fibrous bands of tissue known as adhesions. Adhesions may result in delayed healing, post-operative pain, and/or infection, and potentially a need for further surgical intervention. Thus, there is a need for a barrier for reducing the incidence, extent, and severity of postoperative adhesions to the surgical site.

Adhesion formation can be prevented or limited by providing a physical barrier between the traumatized surgical site and adjacent organs and/or tissue. The physical barrier can be applied to sites of potentially adhesiogenic tissue and organ structures to serve as a temporary barrier separating opposing tissue and/or organ surfaces. These barriers are often provided as films that are applied at the conclusion of the surgical procedure.

While such film-like barriers would be useful, it may be difficult to apply such a barrier during a surgical procedure. For example, during laparoscopic procedures, a surgery is performed using a camera that is mounted on a laparoscope that is inserted through a small surgical port called a trocar. The surgical site is viewed on a monitor and surgical instruments access the site through additional trocars. Conventional trocars vary in diameter from 5mm to 12mm. To minimize trauma and scarring, both the surgeon and patient prefer to use as small a trocar as possible. As a consequence, it becomes challenging to be able to insert a film-like barrier through a trocar and deliver it to a surgical site while viewing the surgical site on a monitor. For example, the film-like barrier will need to be introduced into the body in a collapsed state and then expanded back to its original form. It may be difficult to collapse the barrier (e.g., depending on its material properties, size, and/or shape) and/or expand (e.g., depending on its collapsed orientation/configuration, if it comes into contact with liquid, etc.).

In addition, there are other factors that could complicate the delivery of a film-like barrier in a laparoscopic procedure. For example, due to the confined spaces in laparoscopic surgery, the film-like barrier may adhere to unintended organs and/or tissue. In addition, the film-like barrier may be sticky when it comes in contact with a liquid, such as blood or saline used for irrigation, causing the film-like barrier to inadvertently adhere to the trocar, unintended tissue/organ, or itself. Multiple barriers may be needed to adequately cover the surgical site.

A barrier application device is a secondary medical device that may be used in surgical procedures to move a primary medical device, such as a film-like barrier, into a patient. Existing application devices include a grip, which is often a cylindrical protrusion on or near the handle of the device, and the cylindrical protruding grip typically has a smooth rounded surface. A surgeon may use the grip to control the movement of the application device. However, the smooth rounded surface of the grip may cause the application device to roll on a flat surface, such as a surgical table or tray. Application devices should be easily accessible to a surgeon during a surgical procedure, and the application device must be sterile prior to and during its use during a surgical procedure. For example, an application device must remain in the sterile field during a surgical procedure. Therefore, it is desirable to provide an improved barrier application device, or introducer device, that resists rolling on a flat surface.

### SUMMARY

To improve manufacturability challenges that arise in the design of an introducer device for delivery of film products during a surgical procedure, a new introducer device is described herein. The present disclosure seeks to implement new devices for delivering film barriers to a patient during surgical procedures, and to improve the manufacturability of the devices by simplifying the design and minimizing the overall part count of the introducer device.

In light of the disclosure herein, and without limiting the scope of the invention in any way, in a first aspect of the present disclosure, which may be combined with any other aspect listed herein unless specified otherwise, an introducer device includes a winding rod, an outer cannula assembly, and a molded grip. In an assembled configuration, the winding rod is inserted through the outer cannula assembly. The distal end of the winding rod includes a slit intended to receive a surgical film, and the proximal end of the winding rod includes a handle with a grip. The outer cannula assembly includes a metallic tube with a slit located on its distal end, and a grip located on the proximal end, the grip is located adjacent to the handle of the winding rod.

Accordingly, in a preferred embodiment, the introducer device assembly of the present invention includes three components, the winding rod, the metallic tube, and the molded grip. This simplified design minimizes tooling complexity. Furthermore, the winding rod and the molded grip, in a preferred embodiment, require a simple two-plate mold, or A-B mold, with a single side action, which similarly simplifies the device.

Assembly of the introducer device involves a single glue joint, or optionally, overmolding operation.

In a second aspect, which may be combined with any other aspect described herein, or portion thereof, an introducer device assembly for applying a film at a surgical site comprises a winding rod including a handle coupled to a shaft, wherein the handle is located at a proximal end of the winding rod and includes a plurality of ribs, and the shaft includes a slit located at its distal end, and wherein the shaft of the winding rod has a dumbbell-shaped cross-section; and further comprises an outer cannula assembly including a metallic tube coupled to a molded grip, wherein the molded grip is located at the proximal end of the metallic tube, the metallic tube includes a slit at its distal end and a notch at its proximal end, the notch is configured to mate with a tab on the molded grip for rotational clocking, wherein the molded grip includes at least one substantially flat surface to prevent rolling, and wherein the molded grip includes a plurality of ribs radiating from the surface of the molded grip.

In a third aspect, which may be combined with any other aspect described herein, or portion thereof, the introducer assembly device includes raised protrusions on the handle that are oriented vertically and horizontally

In a fourth aspect, which may be combined with any other aspect described herein, or portion thereof, the introducer device assembly includes a winding rod comprising, in part, a handle that is hollow.

In a fifth aspect, which may be combined with any other aspect described herein, or portion thereof, the introducer assembly device includes a winding rod comprising, in part, a handle that is tapered.

In a sixth aspect, which may be combined with any other aspect described herein, or portion thereof, the introducer assembly device comprises a winding rod including a slit that is adapted to receive a film barrier.

In a seventh aspect, which may be combined with any other aspect described herein, or portion thereof, the introducer assembly device comprises a winding rod including a handle that has an orientation indicator. The orientation indicator may be disposed on the handle of the winding rod. The orientation indicator may be a projection, recess, symbol, or any other feature and may have a color different from a color of the handle to provide visual feedback to the user.

In an eighth aspect, which may be combined with any other aspect described herein, or portion thereof, the introducer assembly device comprises a metallic tube that has an outer diameter of no greater than 4.9 mm.

In a ninth aspect, which may be combined with any other aspect described herein, or portion thereof, the introducer assembly device comprises a metallic tube that has an inner diameter of no less than 4.5 mm.

In a tenth aspect, which may be combined with any other aspect described herein, or portion thereof, the introducer assembly device does not exceed a length of 40 mm.

In an eleventh aspect, which may be combined with any other aspect described herein, or portion thereof, the introducer assembly device is longer than 40 mm.

In a twelfth aspect, any of the features, functionality, and alternatives described in connection with any one or more of FIGS. 1-10 may be combined with any other of FIGS. 1-10.

In light of the above aspects and the present disclosure set forth herein, it is accordingly an advantage of the present disclosure to provide an introducer assembly device for delivering a film material to a patient in need thereof.

It is another advantage of the present disclosure to provide an improved, simplified introducer device with three primary components: winding rod, metallic tube, and molded grip.

It is yet another advantage of the present disclosure to provide an introducer device with a winding rod that has a dumbbell-shaped cross-section to maximize the area moment of inertia to provide resistance to flexion which would otherwise lead to undue deflection in the rod, particularly in the slit distal area of the winding rod.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a perspective view of an assembled introducer device, according to an example embodiment of the present disclosure.

FIG. 2 is a perspective view of the outer cannula assembly, according to an example embodiment of the present disclosure.

FIG. 3 is a perspective view of the metallic tube of the outer cannula assembly, according to an example embodiment of the present disclosure.

FIG. 4 is a perspective view of the winding rod of the introducer device, according to an example embodiment of the present disclosure.

FIGS. 5A-5B are perspective views of the proximal end of the winding rod and the handle, according to an example embodiment of the present disclosure.

FIG. 6A-6B is a foreshortened perspective view and a foreshortened cross-sectional view of the distal portion of the winding rod, respectively, according to an example embodiment of the present disclosure.

FIG. 7 is a detailed view of a cross-section of the winding rod, according to an example embodiment of the present disclosure.

FIGS. 8A-8B are perspective views of a molded grip, according to an example embodiment of the present disclosure.

FIG. 9 is a cross-sectional perspective view of the molded grip, according to an example embodiment of the present disclosure.

FIG. 10 is a schematic illustration of the grip feature of the handle.

FIG. 11 is a schematic illustration of the handle.

FIG. 12 illustrates an alternative embodiment of the molded grip attached to the outer cannula assembly.

### DETAILED DESCRIPTION

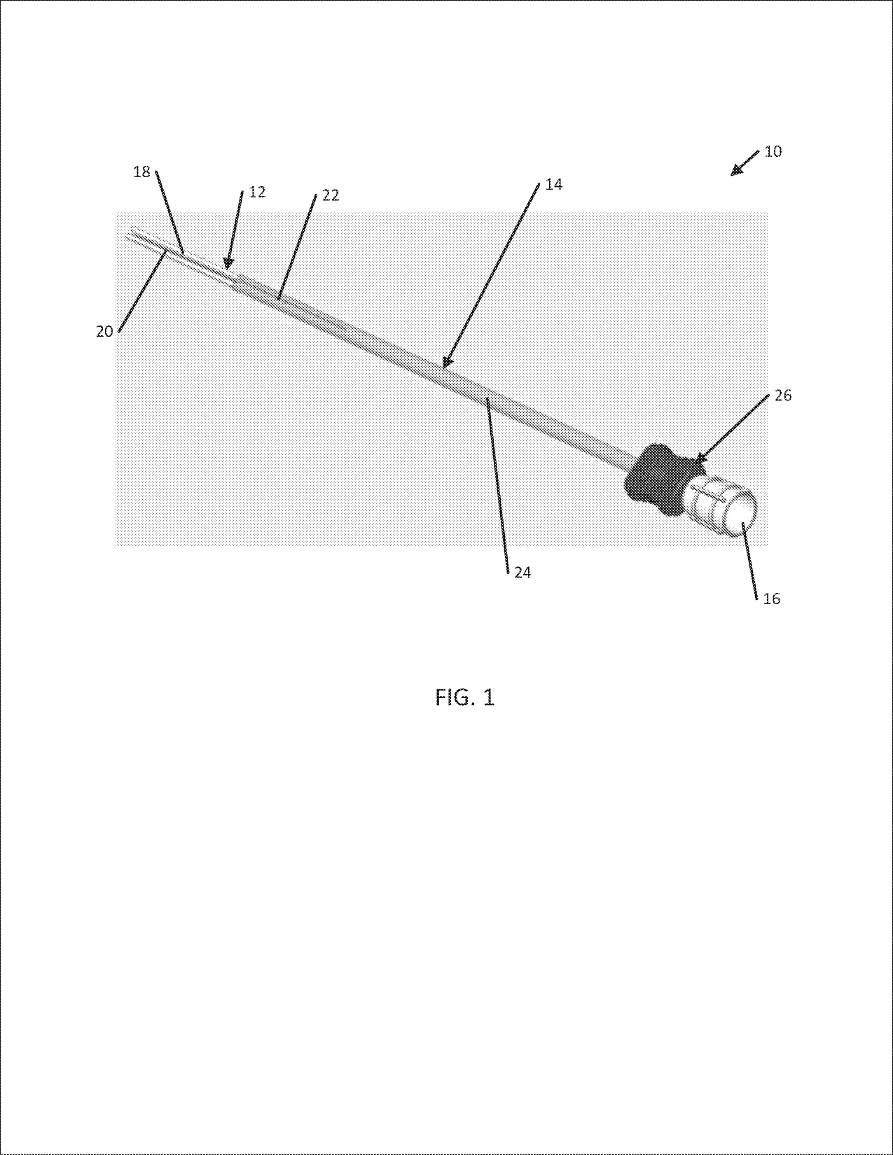

Referring now to the drawings and in particular to FIG. 1, an embodiment of a surgical film barrier introducer device 10 of the present disclosure is illustrated, and which includes a winding rod 12 and an outer cannula assembly 14. The winding rod 12 comprises a handle 16 at the proximal end of the introducer device 10, and a shaft 18 extended therefrom. The distal ends of each of the winding rod 12 and the outer cannula assembly 14 include slits 20, 22, respectively, which are configured to receive a film material. The length of the slits 20, 22 may vary in length to ensure the width of the compatible film products can be accommodated. The winding rod 12 and the outer cannula assembly 14 are initially assembled coaxially such that their slit features are in close proximity to one another. The outer cannula assembly is a subassembly of a metallic tube 24 and a molded grip 26.

During use of the introducer device 10, a user pushes the winding rod 12 to extend beyond the outer cannula assembly 14 and loads film material (not shown) to be delivered to the slit feature 20 on the winding rod 12. Then, the user withdraws the winding rod 12 to align its slit 20 with the slit 22 of the outer cannula assembly 14 and turns the handle 16 of the winding rod 12 to coil the film to be delivered about the winding rod 12, inside the lumen of the outer cannula assembly 14. The introducer device 10 assembly is then introduced into a suitable laparoscopic trocar, or other suitable means of access. After a target position is achieved, the user advances the winding rod 12 to extend beyond the distal end of the outer cannula assembly 14. The residual stress in the film causes the film to naturally uncoil, and the film is available to be grasped and manipulated by the user into position as required. Finally, the user withdraws the introducer device 10 from the patient.

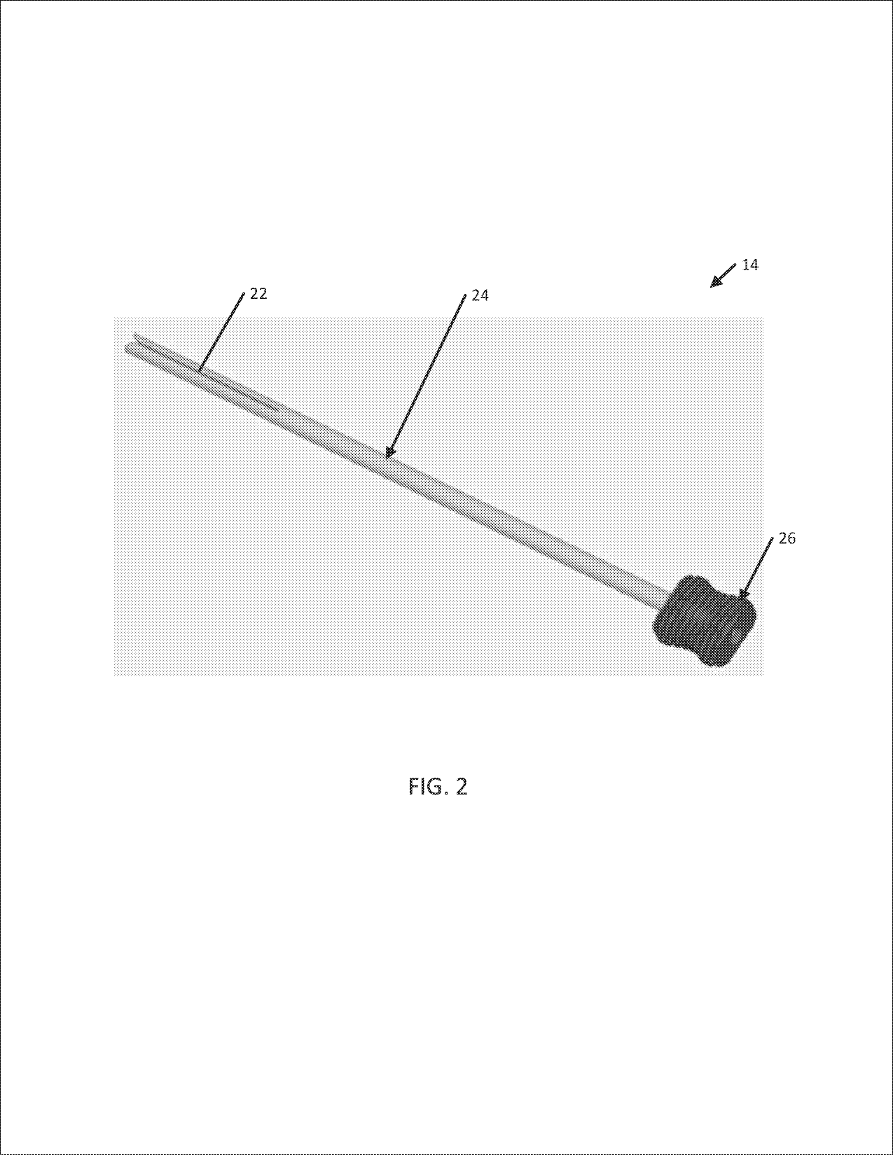

FIG. 2 illustrates the outer cannula assembly 14. As stated above, the outer cannula assembly is a subassembly of a metallic tube 24 and a molded grip 26. The metallic tube 24 and the molded grip 26 may be bonded using a suitable biocompatible adhesive (e.g., cyanoacrylate, epoxy, etc.). Alternatively, the metallic tube 24 and the molded grip 26 may be permanently joined by insert molding.

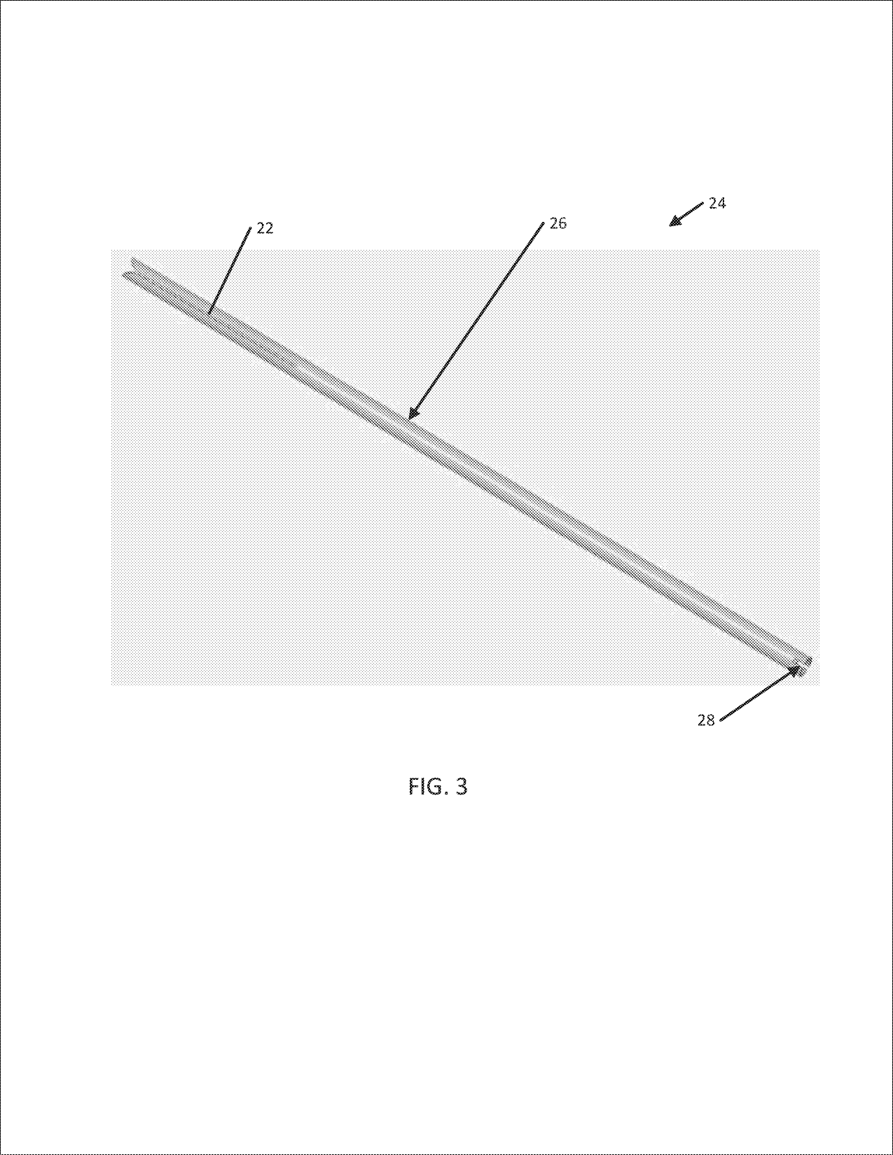

FIG. 3 illustrates the metallic tube 24 of the outer cannula assembly 14 that is detached from the molded grip 26 (not shown). The metallic tube 24 includes a slit 22 disposed on its distal end to provide a location that accommodates a film material as described above in reference to FIG. 1. The slit 22 should be no wider than necessary to maximize part strength.

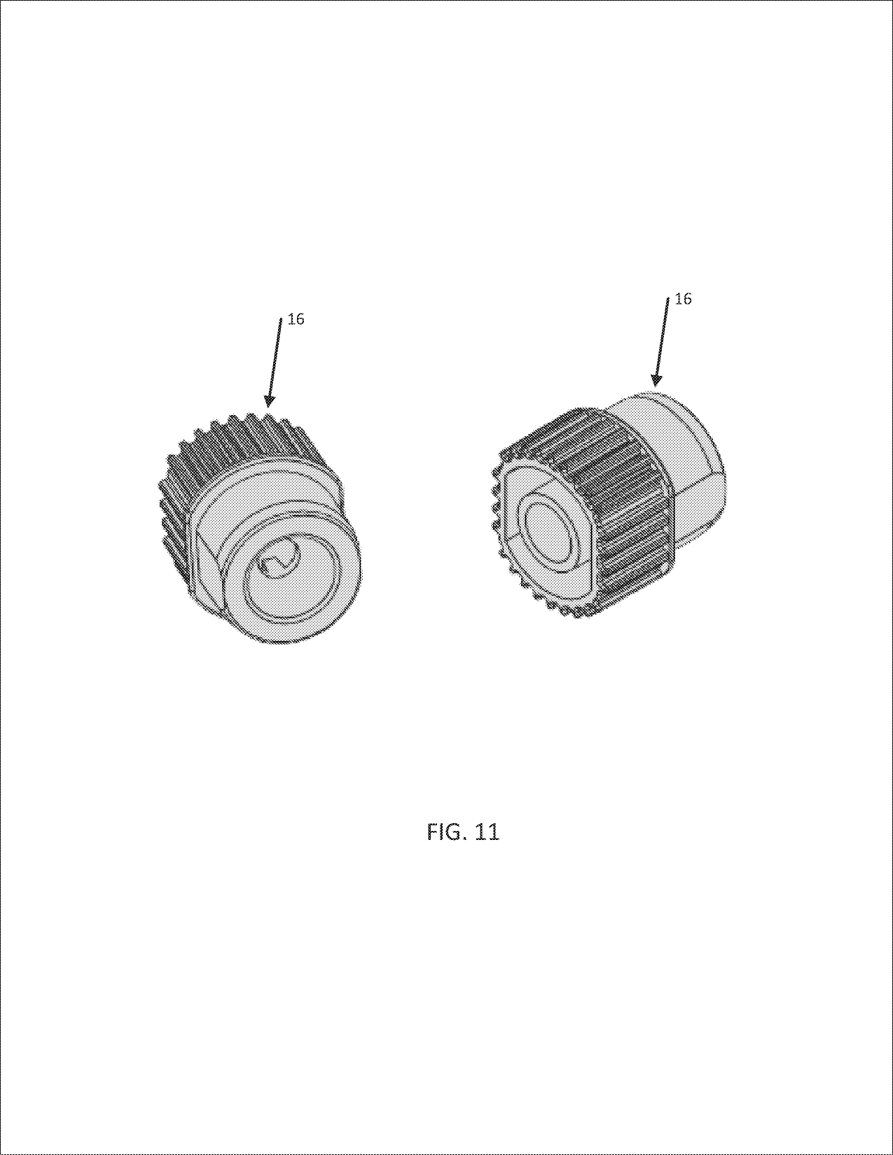

A notch 28 is disposed on the proximal end of the metallic tube 24 to provide a means of rotational clocking. The notch 28 abuts a corresponding tab on the mating molded grip 26 (shown in FIG. 11) to prevent rotation of the metallic tube 24 and the molded grip 26 about one another. The notch 28 may have an approximate width of 1 mm or greater. The slit 22 and the notch 28 may be machined by any suitable means, e.g., laser cutting, abrasive cutting, discharge machining, stamping, etc.

The length of the metallic tube 24 is selected to ensure that the entire introducer device 10 can protrude through a laparoscopic entry port (i.e., a trocar) such that the winding rod’s 12 slit 20, when advanced, completely clears the end of the outer cannula assembly and the trocar itself. This ensures that the film to be delivered can safely unfurl. Under such circumstances, the introducer device 10 may have a length of 40 cm or less. Alternatively, the winding rod 12 and the metallic tube 24 may be of sufficient lengths to provide access to deeper anatomic sites and/or for use during bariatric surgery. Accordingly, the introducer device 10 may have a length of greater than 40 cm.

The metallic tube 24 may have an outer diameter of 4.9 mm or less, which ensures compatibility with virtually all laparoscopic trocars having a nominal diameter of 5 mm. In a preferred embodiment, the metallic tube 24 has an inner diameter of approximately 4.5 mm.

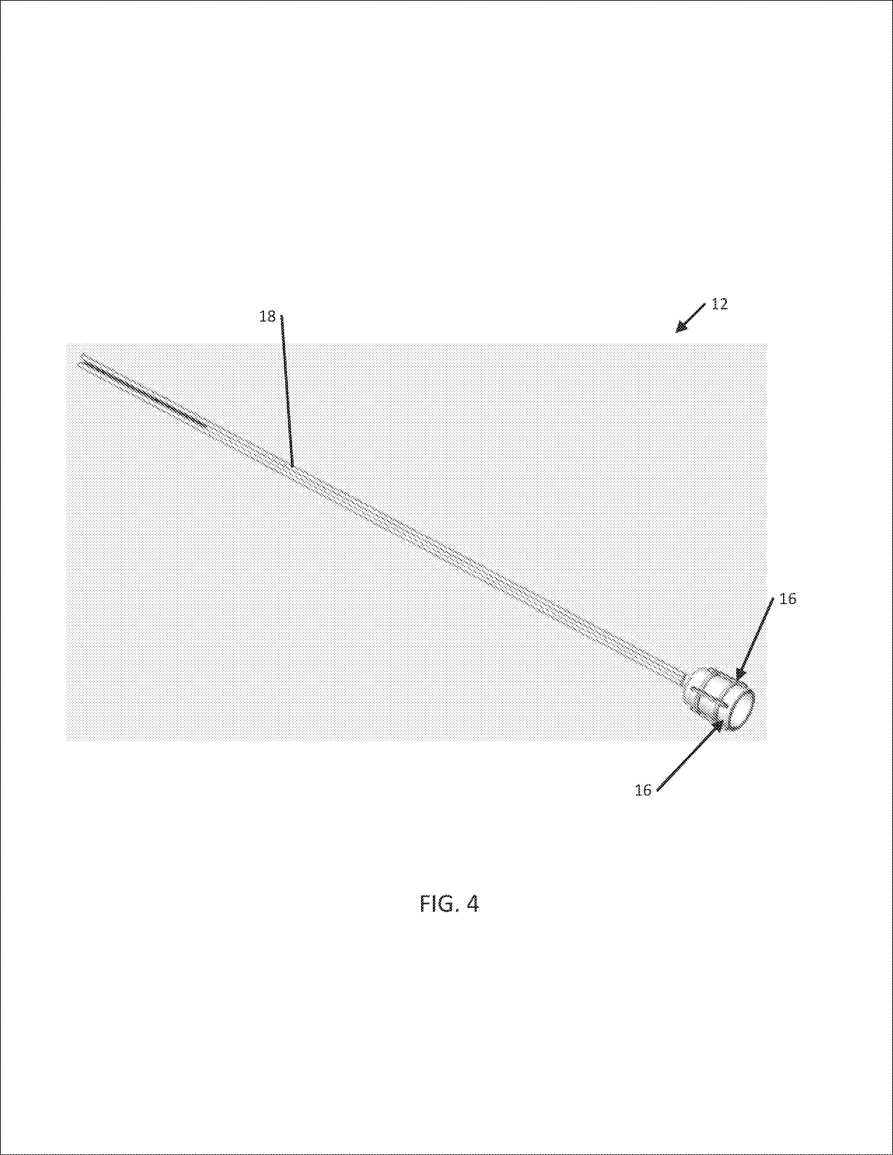

FIG. 4 illustrates the winding rod 12 of the introducer device 10. The winding rod 12 includes a shaft 18 that is in the shape of a dumbbell, which helps wind the film material while also providing sufficient stiffness when the winding rod 12 is rotated. The winding rod 12 includes a handle 16 attached to the shaft 18. The handle 16 is ergonomically shaped (e.g., cylindrical, conical, frusto-conical, or any combination thereof) for being gripped by and handled by a user, and the handle 16 includes a grip feature 29, which may include an array of projections that are disposed on the handle to enhance the grip of the user and prevent slipping during use (as shown in FIG. 1). For example, the grip feature 29 may be beveled (not shown). It should be understood that the grip feature 29 of the handle 16 may include any suitable frictional material.

The winding rod 12 is designed for production by injection molding. It should be understood that any suitable stiff engineering plastic with acceptable biocompatibility and compatibility with the target sterilization modality may be adopted. Examples of suitable materials include nylons, polyether ether ketones (“PEEK”), and acrylonitrile butadiene styrene (“ABS”), optionally with fiber fills to lend additional strength in flexion, torsion, and tension.

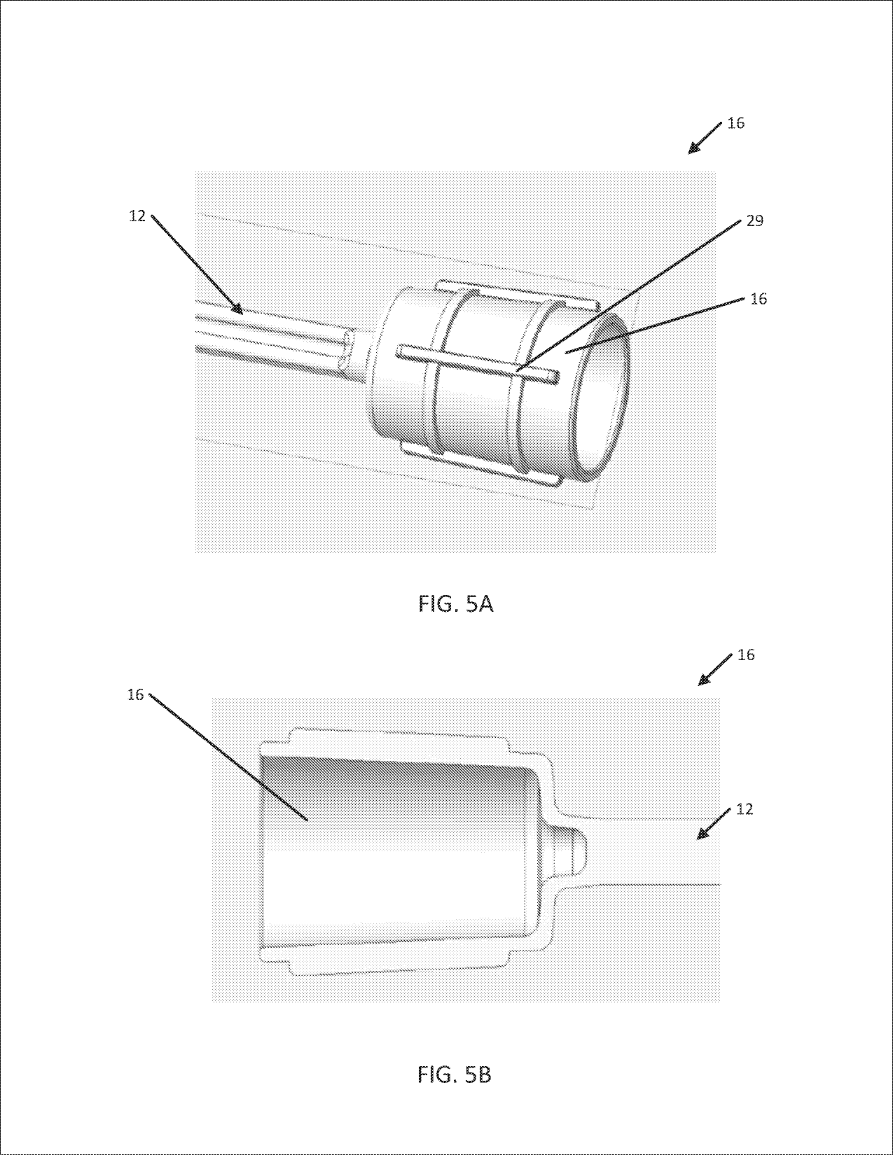

FIGS. 5A-5B illustrate the exterior and interior of the handle 16 located on the proximal end of the winding rod 12. As discussed above in reference to FIG. 2, the winding rod 12 includes a handle 16 that further includes grip features 29, which may include an array of projections that are disposed on the handle to enhance the grip of the user and prevent slipping during use. The grip features 29 may comprise one or more raised protrusions. The raised protrusions may be in any suitable shape or form to increase or enhance the user’s grip. For example, the raised protrusions may be in the shape of an elongated rectangular bar or an elongated bar with tapered ends or rounded ends. The one or more raised protrusions may be disposed on the handle 16 in horizontal and vertical directions. Specifically, the proximal end of the winding rod 12 is slightly tapered and hollow (e.g., the shape of a hollow cone). The slightly tapered, hollow cone shape of the winding rod provides advantages for the manufacture of the winding rod 12, such as ensuring that a consistent, thin wall can be molded using a simple side action. The grip features 29 may include a plurality of ribs 30 on the exterior of the handle 16, which help facilitate turning by a user. Accordingly, the plurality of ribs 30 may be oriented in any suitable manner that enhances the grip of the handle 16, and that avoids undercutting with the parting line A. The handle 16 may be hollow or solid and may be formed by any suitable means. Preferably, the handle 16 is hollow and is formed by a mold side action.

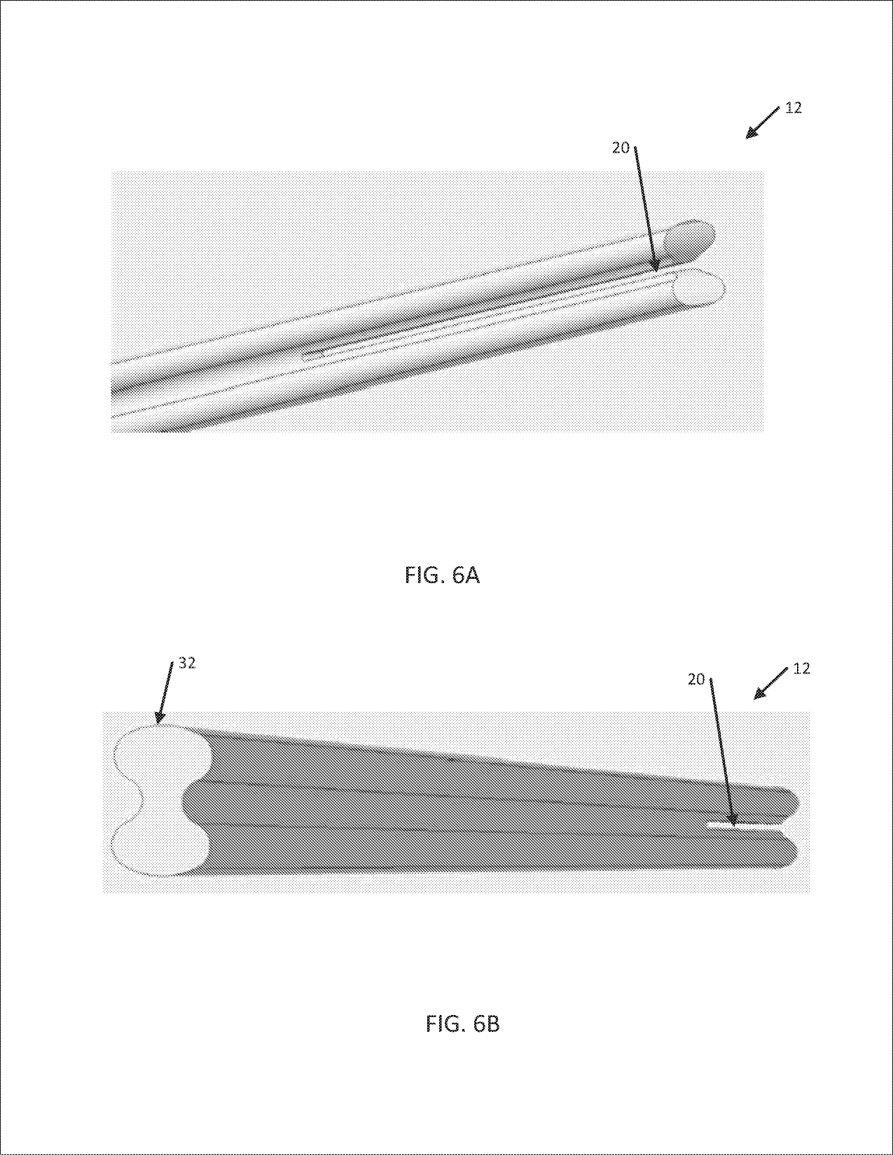

FIGS. 6A-6B illustrate the distal end of the winding rod 12. The bulk of the distal end of the winding rod 16 adopts a dumbbell shaped cross-section 32. The dumbbell shape helps maintain a suitable wall thickness throughout the winding rod 12 and maximizes the area moment of inertia to provide resistance to flexion that would otherwise lead to undue deflection of the winding rod 12, particularly in the slit 20 in the distal end of the winding rod 12.

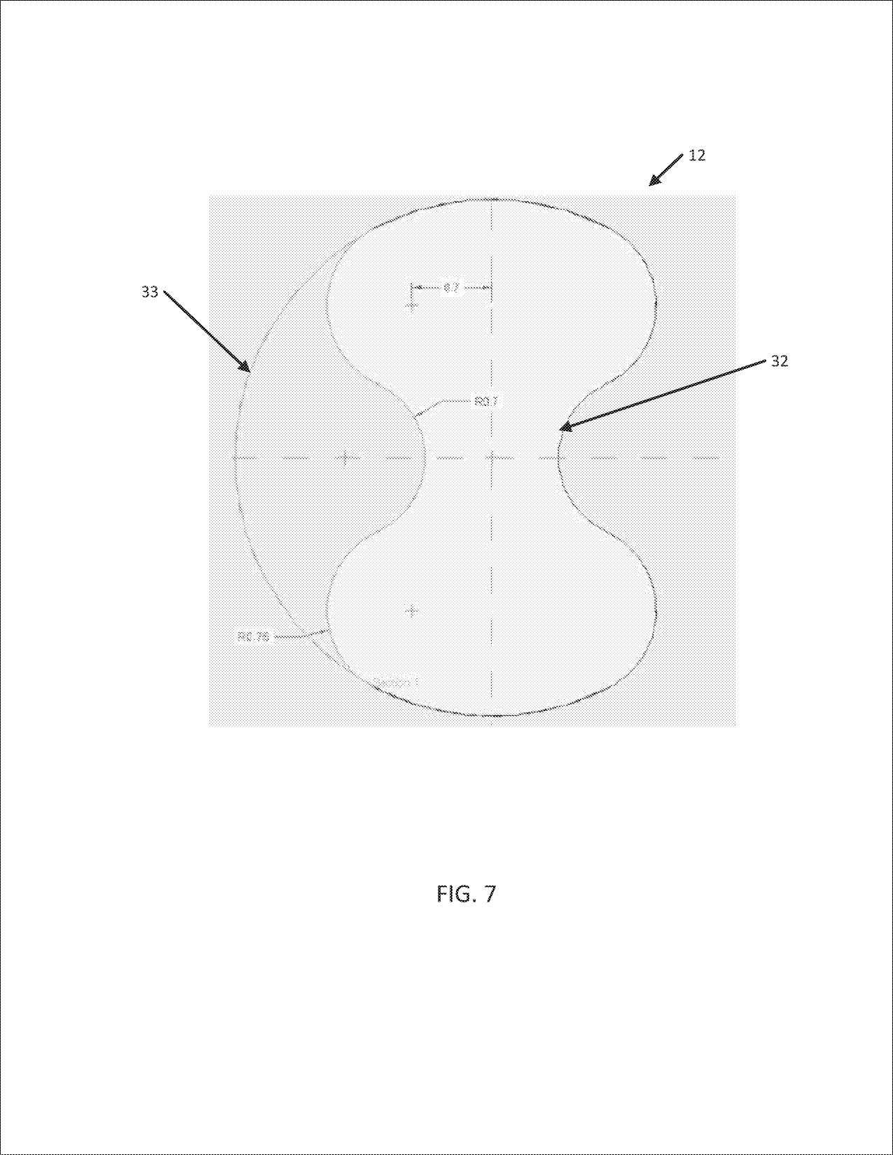

Further, the slit 20 disposed on the distal end of the winding rod 12 is arranged such that it can receive a load of film to be delivered to a patient. The width of the slit 20 should be minimized to maximize part strength, and the length of the slit 20 may be adjusted, e.g., longer or shorter, to accommodate the width of compatible film materials. FIG. 7 is a foreshortened perspective of the cross-section of the winding rod 12, which further illustrates the dumbbell-shaped cross-section 32 of the winding rod 12. More specifically, FIG. 7 provides a diagram detailing the dumbbell-shaped cross-section 32 of the distal end of the winding rod 12. In an example embodiment, the dumbbell-shaped cross-section 32 is circumscribed in a circle 33 with a diameter of 4.5 mm.

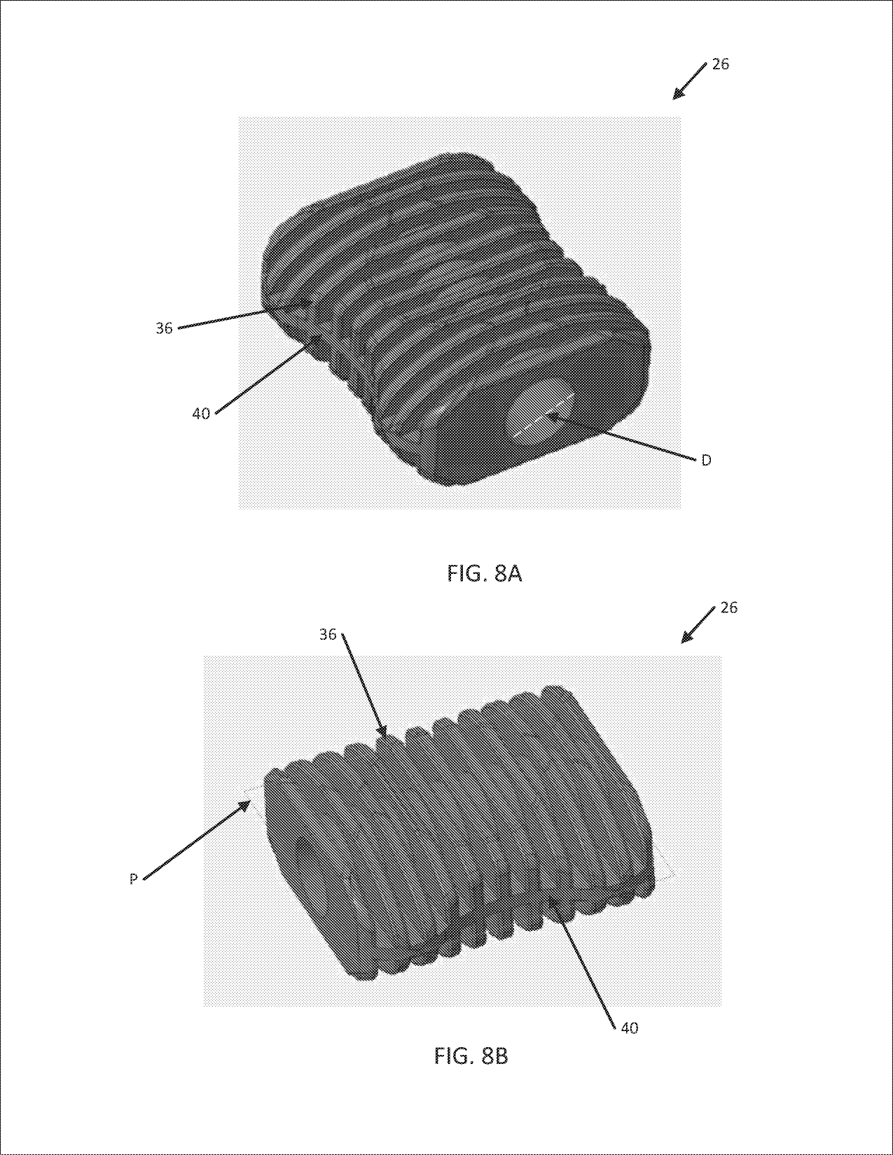

Referring now to FIGS. 8A-8B, a molded grip 26 is illustrated in various angles. The molded grip 26 has an inner hollow diameter (D) suitable to accommodate the outer diameter of the metallic tube 24 of the outer cannula assembly 14 via a clearance fit. The shape of the molded grip 26 may be any suitable shape that prevents the introducer device 10 from rolling on a flat surface, and the shape should provide a generally smooth but graspable surface for manipulation of the tool in minimally invasive and/or laparoscopic surgery. For example, the molded grip 26 may be generally in the shape of a rectangle or square with or without concave outer edges running the length of the molded grip 26, or the molded grip 26 may be generally in an oblong shape with at least one flat surface, cylindrical with at least one flat surface, etc. The surface of the molded grip 26 includes a plurality of ribs 36 that radiate from the flat surface of the molded grip 26 that is coincident with the parting line 40 on the molded grip 26. The ribs 36 and the overall form of the molded grip 26 are formed by the cavity and core sides of the mold of the molded grip 26. A preferential parting line 40 is indicated by plane P, which runs directly through the center of the length of the molded grip 26.

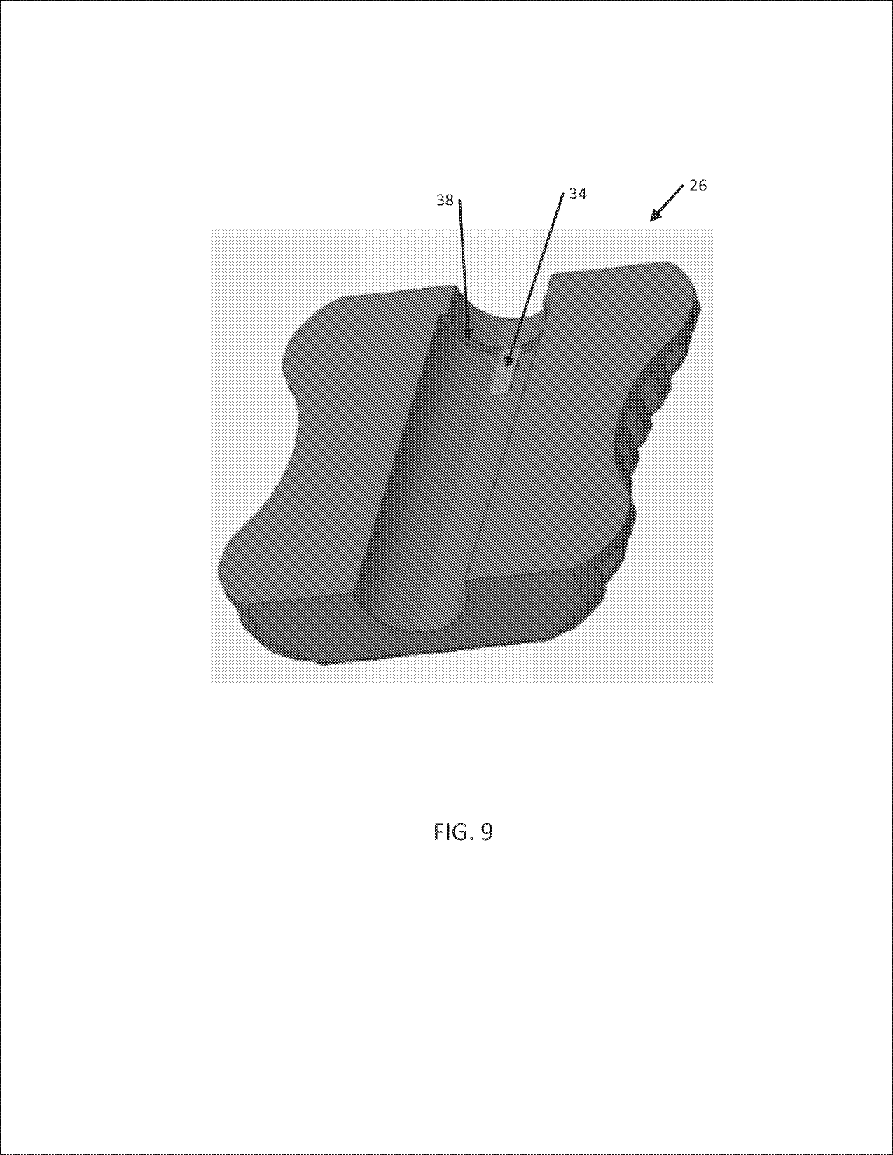

FIG. 9 is a cross-section illustration of the interior of the molded grip 26. On the interior of the molded grip 26 is at least one tab 34 that mates with the notch 28 on the metallic tube 24 of the outer cannula assembly 14 (shown in FIG. 3), which locks the molded grip 26 and the metallic tube 24 during assembly, and prevents slipping, rotation, and/or general failure in torsion that would separate the molded grip 26 and the metallic tube 24 during use. The molded grip 26 may include a plurality of tabs 34 that appear on the interior of the molded grip 26 in a uniform pattern about the central axis of the molded grip 26. The tab 34 may abut against a vertically protruding edge 38. The vertically raised edge 38 may extend around the entire circumference of the hollow inside of the molded grip 26. The interior of the molded grip 26 is formed by a side action.

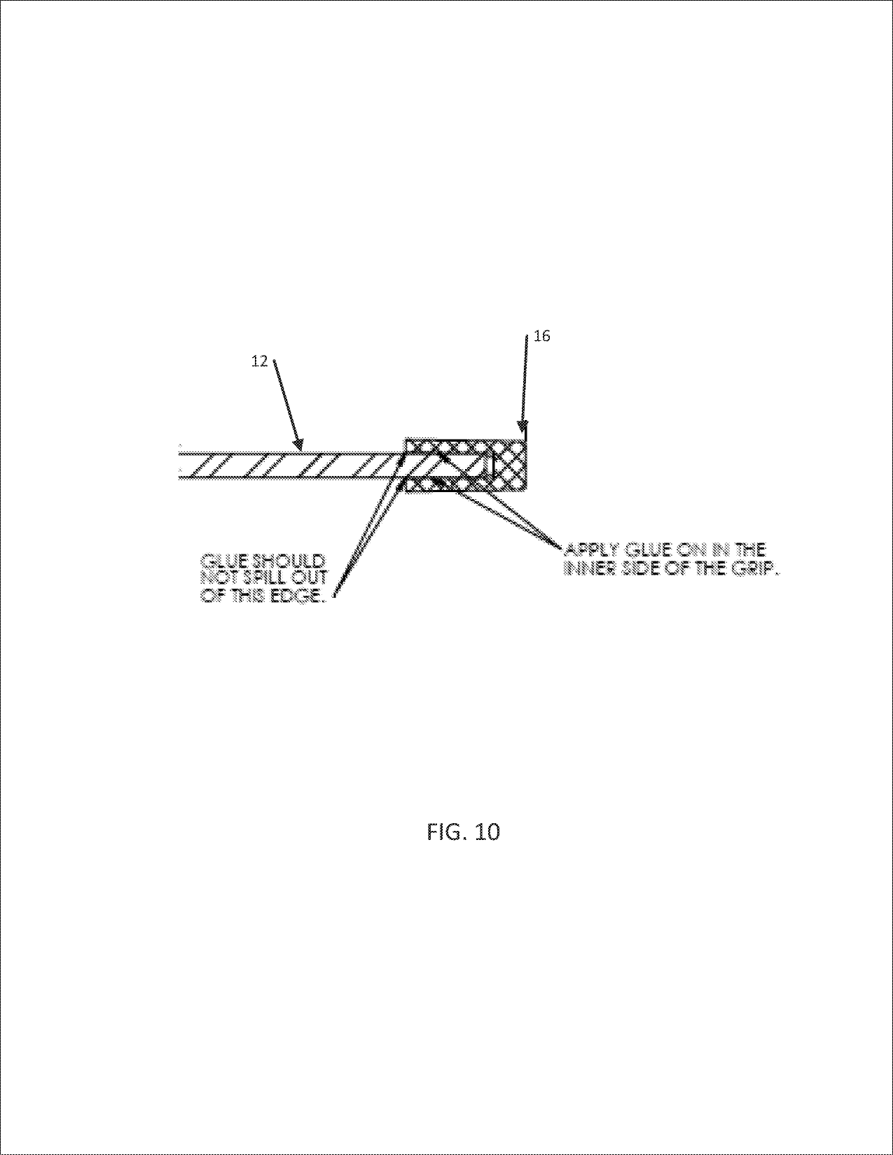

FIG. 10 is a schematic illustration of the grip feature of the handle. The grip feature 29 is permanently affixed to the winding rod 12 by any suitable means. For example, the grip feature 29 may be glued to the winding rod 12.

FIG. 11 is a schematic illustration of the handle. The handle 16 may include at least one substantially flat side. Further, the handle 16 and may include grip features, such as a plurality of ridges to increase friction with the user’s fingers.

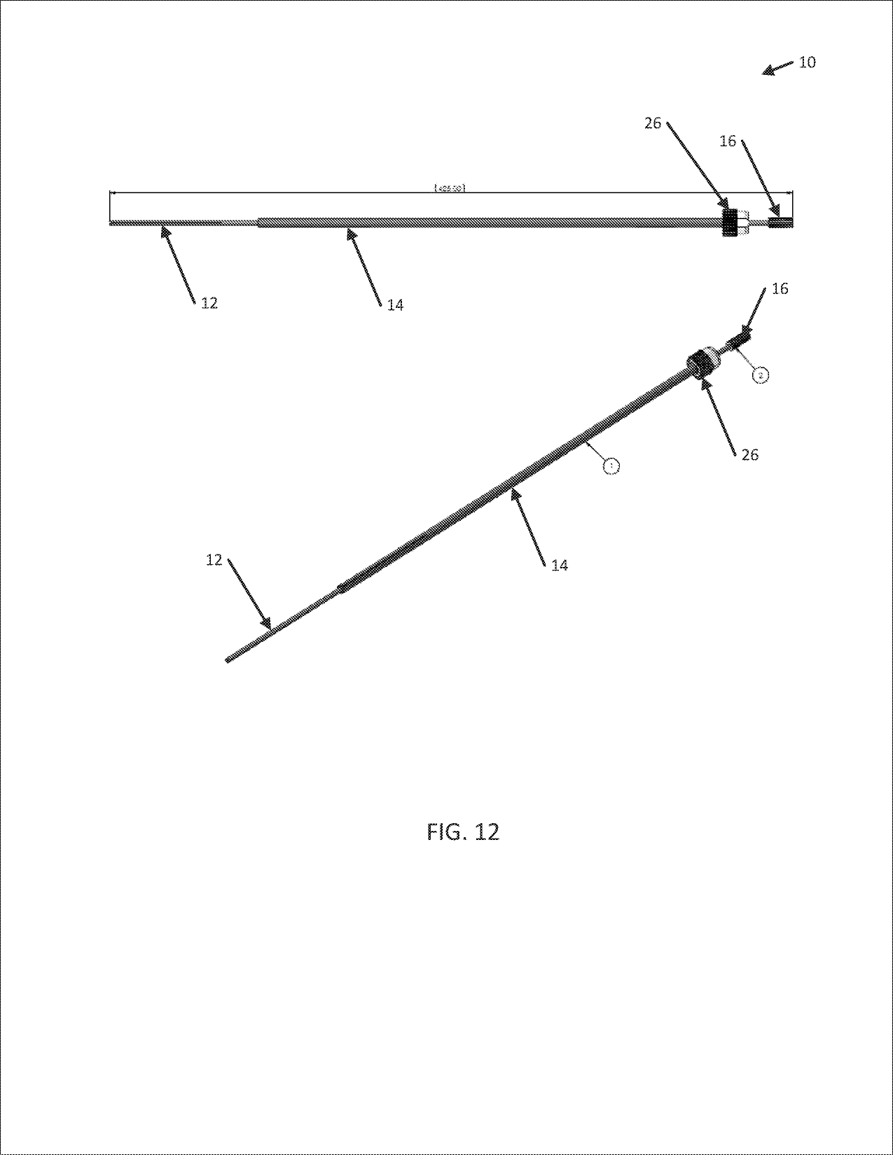

FIG. 12 illustrates an alternative embodiment of the molded grip attached to the outer cannula assembly. The molded grip 26 is partially cylindrical in shape and is attached to the outer cannula assembly 14. The molded grip 26 may include at least one flat side to prevent the introducer device 10 from rolling on flat surfaces.

While the exemplary embodiments of the introducer device have been described with respect to a film barrier, those of skill in the art will understand that other materials may be retained and deployed by the application device according to the present invention. For example, gauze, a sponge, or any other thin, flexible material may be retained and deployed by the application device of the present invention in a manner similar to as described above with respect to a film barrier.

Those of skill in the art will understand that modifications (additions and/or removals) of various components of the apparatuses, methods and/or systems and embodiments described herein may be made without departing from the full scope of the present invention, which encompass such modifications.

1. An introducer assembly for applying a film at a surgical site, the introducer assembly comprising:
a winding rod including a handle coupled to a shaft, wherein the handle is located at a proximal end of the winding rod and includes a plurality of raised protrusions, and the shaft includes a slit located at its distal end, and wherein the shaft of the winding rod has a dumbbell-shaped cross-section; and
an outer cannula assembly including a metallic tube coupled to a molded grip, wherein the molded grip is located at the proximal end of the metallic tube, the metallic tube includes a slit at its distal end and a notch at its proximal end, the notch is configured to mate with a tab on the molded grip for rotational clocking, wherein the molded grip includes at least one substantially flat surface to prevent rolling, and wherein the molded grip includes a plurality of ribs radiating from the surface of the molded grip.

2. The introducer assembly according to claim 1, wherein the raised protrusions on the handle are oriented vertically and horizontally.

3. The introducer assembly according to claim 1, wherein the handle of the winding rod is hollow.

4. The introducer assembly according to claim 1, wherein the handle of the winding rod is tapered.

5. The introducer assembly according to claim 1, wherein the winding rod includes a slit that is adapted to receive a film barrier.

6. The introducer assembly according to claim 1, wherein the handle includes an orientation indicator.

7. The introducer assembly according to claim 1, wherein the metallic tube has an outer diameter of no greater than 4.9 mm.

8. The introducer assembly according to claim 1, wherein the metallic tube has an inner diameter of no less than 4.5 mm.

9. The introducer assembly according to claim 1, wherein a length of the introducer assembly does not exceed 40 mm.

10. The introducer assembly according to claim 1, wherein a length of the introducer assembly is longer than 40 mm.
